(19) United States
(12) Patent Application Publication
(10) Pub. No.: US 2023/0375489 A1
(43) Pub. Date: Nov. 23, 2023

(54) GAS SENSOR

(71) Applicant: NGK SPARK PLUG CO., LTD, Nagoya-shi, Aichi (JP)

(72) Inventors: Masahiro YAMASHITA, Nagoya-shi, Aichi (JP); Shoji KITANOYA, Nagoya-shi, Aichi (JP); Yusuke MATSUKURA, Nagoya-shi, Aichi (JP)

(73) Assignee: NGK SPARK PLUG CO., LTD, Nagoya-shi, Aichi (JP)

(21) Appl. No.: 18/028,321

(22) PCT Filed: Sep. 15, 2021
(86) PCT No.: PCT/JP2021/033877

§ 371 Date: Mar. 24, 2023

(30) Foreign Application Priority Data
Nov. 26, 2020 (JP) 2020-195836

(51) Int. Cl. G01N 25/18; G01N 33/00

(52) U.S. Cl. CPC G01N 25/18; G01N 33/0027

(57) ABSTRACT

A gas sensor 1 according to the present invention includes: a gas sensor element 22; a box-like casing 3 having a wall portion 3B1 provided with a gas introduction port 3B2; a circuit board 4 having a mounting surface 4a on which the gas sensor element 22 is mounted, and disposed inside the casing 3 such that the mounting surface 4a is away from the wall portion 3B1 and the gas sensor element 22 overlaps with the gas introduction port 3B2; and an annular elastic seal body 5 formed on the mounting surface 4a so as to surround the gas sensor element 22, and disposed between the mounting surface 4a and the wall portion 3B1. The elastic seal body 5 is made of sulfur-free condensation silicone resin.

### TECHNICAL FIELD

The present invention relates to a gas sensor.

### BACKGROUND ART

A gas sensor for detecting the concentration of a to-be-detected gas (e.g., an inflammable gas such as hydrogen) has been known. For example, as shown in Patent Literature 1, this type of gas sensor has such a configuration that a circuit board on which a gas sensor element is mounted for detecting a to-be-detected gas is housed in a box-like casing made of resin. An annular elastic seal body is interposed between the circuit board and the casing so as to surround the gas sensor element. The elastic seal body, the circuit board, and the casing form a sealed space. When an atmospheric gas containing a to-be-detected gas is introduced from a gas introduction port provided in the casing into the space, the concentration of the to-be-detected gas in the space is detected by the gas sensor element.

An example of the material to be used for forming the elastic seal body is a rubber material such as an ethylene-propylene rubber (EPDM). Vulcanization (sulfur crosslinking) is generally performed on the elastic seal body for the purpose of improving the rubber elasticity or the like.

### CITATION LIST

Patent Literature 1:
Japanese Unexamined Patent Application Publication No. 2017-122616

### Technical Problem

When a sulfur component is contained in the elastic seal body due to vulcanization or the like, a corrosive component (e.g., a sulfur component such as sulfur gas) containing sulfur may be generated from the elastic seal body with time and may corrode pad parts on which the gas sensor element formed on the circuit board is mounted.

The pad parts contain copper (Cu), and thus, when the pad parts react with the corrosive component, copper sulfide (CuS) is generated. For example, if the copper sulfide grows so as to connect the adjacent pad parts to each other, a short circuit between the terminals of the gas sensor element may occur, resulting in malfunction of the gas sensor element.

### SUMMARY OF INVENTION

An object of the present invention is to provide a gas sensor in which a corrosive component containing sulfur is prevented from being generated from an elastic seal body surrounding a gas sensor element.

### Solution to Problem

The means for solving the problem is as follows.

<1> A gas sensor including: a gas sensor element; a box-like casing having a wall portion provided with a gas introduction port; a circuit board having a mounting surface on which the gas sensor element is mounted, and disposed inside the casing such that the mounting surface is away from the wall portion and the gas sensor element overlaps with the gas introduction port; and an annular elastic seal body formed on the mounting surface so as to surround the gas sensor element, and disposed between the mounting surface and the wall portion, wherein the elastic seal body is made of sulfur-free condensation silicone resin.

<2> The gas sensor according to the <1>, wherein the gas sensor element is composed of a thermal conductivity gas sensor element including a heating resistor having a resistance value that changes according to change in a temperature of the gas sensor element.

### Advantageous Effects of Invention

The present invention can provide a gas sensor in which a corrosive component containing sulfur is prevented from being generated from an elastic seal body surrounding a gas sensor element.

### BRIEF DESCRIPTION OF DRAWINGS

FIG. 1 is a sectional view schematically illustrating the configuration of a gas sensor according to Embodiment 1.

FIG. 2 is an enlarged sectional view of a portion around a measurement chamber in FIG. 1.

FIG. 3 is a diagram illustrating the arrangement relation between pad parts and an elastic seal body on a circuit board.

### DESCRIPTION OF EMBODIMENTS

### Embodiment 1

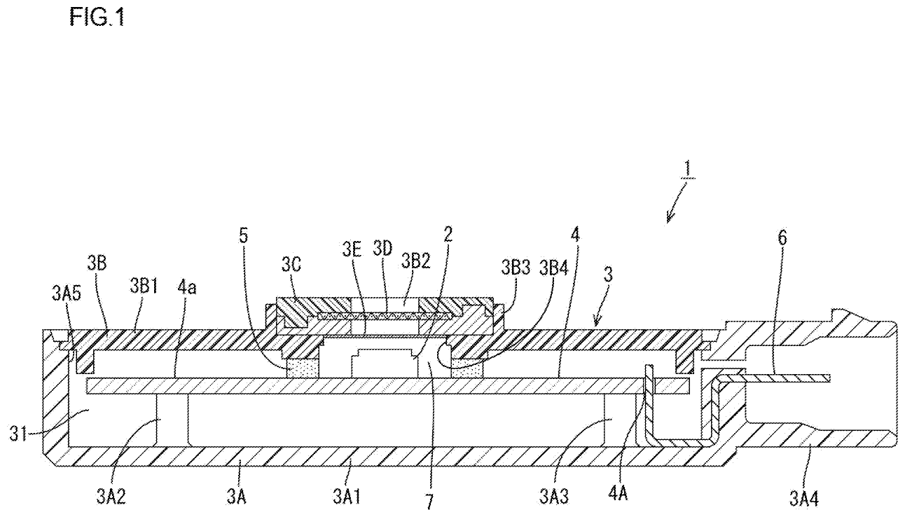

Embodiment 1 of the present invention will be described with reference to FIG. 1 to FIG. 3. FIG. 1 is a sectional view schematically illustrating the configuration of a gas sensor 1 according to Embodiment 1. For the convenience of description, the upper side in FIG. 1 is taken as the upper side of the gas sensor 1, and the lower side in FIG. 1 is taken as the lower side of the gas sensor 1. The gas sensor 1 is a device for detecting the concentration of a to-be-detected gas by utilizing thermal conductivity of the same. Such a gas sensor 1 is, for example, disposed in a system (e.g., a fuel cell vehicle or a residential fuel cell system) equipped with a fuel cell using hydrogen gas as an energy source, and detects hydrogen gas which is an inflammable gas. Accordingly, leakage of the hydrogen gas can be detected in the system.

The gas sensor 1 mainly includes a detection element assembly 2, a casing 3, a circuit board 4, and an elastic seal body 5, as shown in FIG. 1.

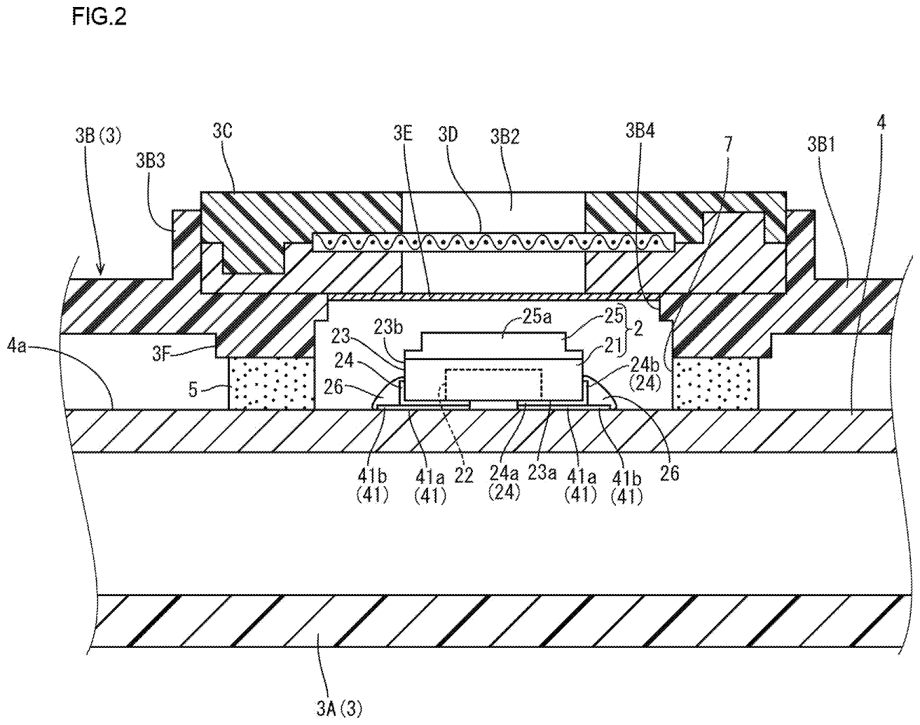

FIG. 2 is an enlarged sectional view of a portion around a measurement chamber. The detection element assembly (capped package) 2 includes a gas sensor package 21 and a protective cap 25, as shown in FIG. 2.

The gas sensor package 21 mainly includes a gas sensor element 22, a body part 23, and a plurality of terminal parts 24. The gas sensor element 22 is a thermal conductivity detection element (thermal conductivity gas sensor element) including a heating resistor (not shown) having a resistance value that changes according to change in the temperature of the gas sensor element 22. The body part 23 is a box-like member opened upward to house the gas sensor element 22, and has a substantially rectangular parallelepiped shape. The body part 23 is made of an insulating ceramic. Each terminal part 24 is a conductive member connected to an electrode of the gas sensor element 22 in the body part 23, and has a bottom-side terminal portion 24a disposed on a bottom surface 23a of the body part 23 and a side-face-side terminal portion 24b rising from the bottom-side terminal portion 24a and formed on an outer side surface 23b of the body part 23.

The protective cap 25 is a lid member disposed so as to cover the opening of the box-like body part 23, and is shaped so as to rise convexly toward the upper side. A plurality of vents (not shown) for introducing an atmospheric gas including a to-be-detected gas into the inside of the protective cap 25 are formed in an upper surface portion 25a of the protective cap 25. In the body part 23, formation positions of these vents are set so as not to overlap, in the up-down direction, with a position where the heating resistor of the gas sensor element 22 is disposed. Such set positions of the vents prevent foreign matter such as dust from approaching the heating resistor even if the foreign matter enters the inside of the protective cap 25. If the heat generated by the heating resistor has been transferred to the foreign matter, the accuracy of detecting the concentration of a to-be-detected gas is lowered. Thus, the protective cap 25 is mounted to the body part 23 of the gas sensor package 21 as described above.

The casing 3 is a substantially box-like member for housing the detection element assembly 2, the circuit board 4, and the like, and is made of non-conductive resin. The casing 3 includes therein a space 31 for housing the detection element assembly 2 and the like. The casing 3 includes a first casing part 3A and a second casing part 3B. The space 31 is surrounded by the first casing part 3A and the second casing part.

The first casing part 3A is of a container shape opened upward, as a whole, and has a wall portion 3A1 which forms a bottom side (lower side) thereof provided with convex support portions 3A2, 3A3 for supporting the circuit board 3. In addition, a connector portion 3A4 is disposed on a lateral side (right side in FIG. 1) of the first casing part 3A. A portion opened upward of the first casing part 3A is referred to as opening 3A5.

The second casing part 3B has a lid (plate) shape to cover the opening 3A5 of the first casing part 3A, and has a wall portion 3B1 provided with a gas introduction port 3B2. The gas introduction port 3B2 is for introducing a to-be-detected gas into the measurement chamber 7, and is provided substantially at the center of the second casing part 3B. In the gas introduction port 3B2, a net member 3D made of metal is disposed.

The wall portion 3B1 of the second casing part 3B is provided with an annular member 3B3 projecting upward in an annular shape, and a frame part 3C for holding the net member 3D is housed on an inner side of the annular member 3B3. A hole penetrating in the up-down direction is provided on an inner side of the frame part 3C, and is used as the gas introduction port 3B2. The frame part 3C is formed by two parts to hold the net member 3D from above and below. In another embodiment, the net member and the frame part may be integrally formed by insert molding or the like to be formed as one part.

The wall portion 3B1 of the second casing part 3B is provided with an opening 3B4 that is provided so as to overlap with the gas introduction port 3B2, and a water-repellent filter 3E is provided so as to cover the opening 3B4 and overlap with the gas introduction port 3B2. In the present specification, the annular member 3B3 and the frame part 3C which holds the net member 3D are also formed as a part of the wall portion 3B1 of the second casing part 3B.

The net member 3D is formed from, for example, a mesh-like wire net, and has a function (flame arrestor function) of preventing flame from going outside the gas sensor 1 even if the heating resistor (described below) of the gas sensor element included in the gas sensor package 21 is heated to high temperatures and an inflammable to-be-detected gas ignites. The water-repellent filter 3E has a function of preventing water from entering the casing 3 from the gas introduction port 3B2. The to-be-detected gas can pass through the water-repellent filter 3E.

The connector portion 3A4 disposed in the first casing part 3A is used for electric connection with an external circuit. A plurality of connector pins 6 are provided inside the connector portion 3A4. The circuit board 3 has a plurality of through holes 4A for connecting the plurality of connector pins 6. Each connector pin 6 is fixed on the circuit board 34 by soldering in a state where the connector pin 6 is inserted in the through hole 4A.

The circuit board 4 is a board including a circuit for detecting the concentration of the to-be-detected gas, and the detection element assembly 2 is mounted on a surface (mounting surface) 4a that is the upper side of the circuit board 4. Besides the detection element assembly 2, a microcomputer for controlling the gas sensor element and a variety of electronic components (not shown) are mounted on the circuit board 4 by soldering or the like.

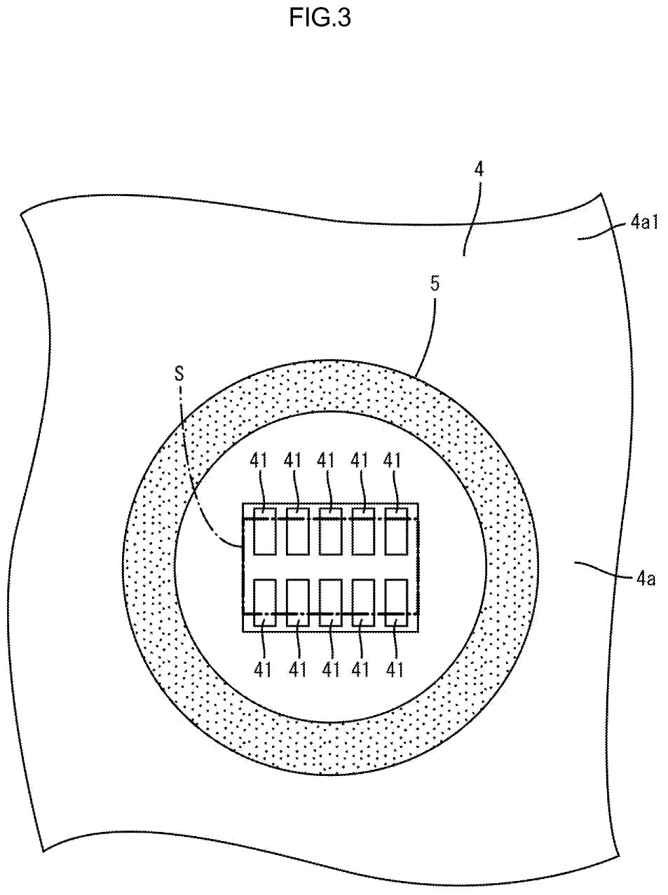

In addition, a plurality of pad parts 41 to be used for mounting the detection element assembly 2 are disposed on the mounting surface 4a of the circuit board 4. FIG. 3 is a diagram illustrating the arrangement relation between the pad parts 41 and the elastic seal body 5 on the circuit board 4. The pad parts 41 are each a thin layered member made of copper or a copper alloy and are formed on the circuit board 4 by a known method (etching or the like). Besides the pad parts 41, pattern-like wiring or the like, which is not shown, is formed on the circuit board 4.

The detection element assembly 2 is disposed on the circuit board 4 such that the plurality of terminal parts 24 disposed on the body part 23 respectively overlap with the corresponding pad parts 41. Upon mounting, paste-like solder is applied on the pad parts 41, and the bottom-side terminal portions 24a of the terminal parts 24 are placed on the solder and reflow is performed in such a state.

The pad parts 41 are larger than the bottom-side terminal portions 24a, and each have a portion (extended portion 41b) protruding outside a portion (overlapping portion 41a) where the bottom-side terminal portion 24a and the pad part 41 are overlapped. To electrically connect the protruding extended portions 41b to the side-face-side terminal portions 24b of the terminal part 24, soldering is performed. Accordingly, solder parts 26 are formed so as to cover the extended portions 41b and the side-face-side terminal portions 24b.

As described above, the gas sensor element 22 is housed in the gas sensor package 21 of the detection element assembly 2 and is mounted on the mounting surface 4a of the circuit board 4 in such a state. Accordingly, it is sometimes expressed as, “the gas sensor element 22 is mounted on the mounting surface 4a of the circuit board 4” in the present specification.

As shown in FIG. 1, the circuit board 4 is supported by a plurality of the support portions 3A2, 3A3 provided on the bottom side of the container-shaped first casing part 3A, and is housed in the casing 3 in such a state. In the casing 3, the mounting surface 4a of the circuit board 4 is away from the wall portion 3B1 of the first casing part 3A, and the detection element assembly 2 mounted on the circuit board 4 is disposed so as to overlap with the gas introduction port 3B2 in the up-down direction. The elastic seal body 5 is interposed between such a circuit board 4 and such a wall portion 3B1.

An annular portion 3F rising annularly toward the lower side (circuit board 4 side) is provided on the side, of the wall portion 3B1, opposed to the mounting surface 4a of the circuit board 4, and the elastic seal body 5 is interposed between the annular portion 3F and the mounting surface 4a of the circuit board 4. The annular portion 3F is formed as a part of the wall portion 3B1. The opening 3B4 is provided on an inner side of the annular portion 3F.

A space surrounded by the circuit board 4, the elastic seal body 5, and the wall portion 3B1 of the casing 3 is used as the measurement chamber 7 for housing an atmospheric gas introduced through the gas introduction port 3B2 from outside, to detect the concentration of a to-be-detected gas.

The elastic seal body 5 is made of sulfur-free condensation silicone resin. The sulfur-free condensation silicone resin means condensation silicone resin not containing sulfur, and commercially available products can be used. In the present specification, “sulfur-free” means having a sulfur content of 50 ppm or less, as a result of an analysis according to the electric furnace combustion method specified by JIS K 6233-3. For the sulfur-free condensation silicone resin, a curing reaction proceeds while absorbing moisture contained in the atmosphere. Thus, the sulfur-free condensation silicone resin does not need heating during the curing process, and can be cured at normal temperature (room temperature). The sulfur-free condensation silicone resin releases small molecules (e.g., acetone or the like) during the curing reaction. However, such small molecules are not substantially released from the cured sulfur-free condensation silicone resin, and thus the elastic seal body 5 has no influence on the accuracy of detecting the concentration of a to-be-detected gas.

In addition, the sulfur-free condensation silicone resin to be used for the elastic seal body 5 has, in a cured state, appropriate elasticity that allows the sulfur-free condensation silicone resin to be used as a substitute for the conventional elastic seal body.

The sulfur-free condensation silicone resin may be a one-component type for which a condensation reaction proceeds in a single liquid, or a two-component type for which a condensation reaction proceeds in a state where a main agent and a curing agent are mixed. From the viewpoint of handleability, workability, and the like, the one-component type of sulfur-free condensation silicone resin is preferable.

The elastic seal body 5 is, for example, produced by the following method. First, the sulfur-free condensation silicone resin (hereinafter, sometimes referred to as sulfur-free condensation silicone resin composition) having flowability in an uncured state is applied annularly on the mounting surface 4a of the circuit board 4 by using a known coater such as a dispenser, and the wall portion 3B1 (annular portion 3F) of the casing 3 is brought into contact with the applied matter such that the applied matter is interposed between the mounting surface 4a of the circuit board 4 and the annular portion 3F of the casing 3. Then, the applied matter (sulfur-free condensation silicone resin composition) interposed between the mounting surface 4a of the circuit board 4 and the annular portion 3F of the casing 3 is left at normal temperature for a predetermined time to allow the sulfur-free condensation silicone resin composition to be cured. In this way, the elastic seal body 5 made of a cured product (sulfur-free condensation silicone resin) of the sulfur-free condensation silicone resin composition is obtained.

In another embodiment, unlike the above-described method, first, the sulfur-free condensation silicone resin composition may be applied on the wall portion 3B1 (annular portion 3F) of the casing 3. Then, the mounting surface 4a of the circuit board 4 may be brought into contact with the applied matter, and the applied matter may be naturally cured.

The sulfur-free condensation silicone resin has adhesiveness, and exerts adhesiveness to the wall portion 3B1 of the casing 3 and the mounting surface 4a of the circuit board 4 when cured from an uncured state. Thus, a gap is prevented from being formed between the elastic seal body 5 and the wall portion 3B1 (annular portion 3F) of the casing 3 and between the elastic seal body 5 and the mounting surface 4a of the circuit board 4. Accordingly, the measurement chamber 7 has excellent airtightness (sealability).

Since the elastic seal body 5 of the present embodiment contains no sulfur, a corrosive component (e.g., a sulfur component such as sulfur gas) containing sulfur is prevented from being generated from the elastic seal body 5. Thus, the pad parts 41 formed on the circuit board 4 are prevented from being corroded by a corrosive component.

The gas sensor 1 of the present embodiment prevents a short circuit between the terminal parts 24 of the gas sensor element 22 that is caused by a corrosion product (copper sulfide) forming so as to connect the adjacent pad parts 41 to each other and that results in malfunction of the gas sensor element.

As shown in FIG. 3, the pad parts 41 on the mounting surface 4a of the circuit board 4 are disposed near the elastic seal body 5. In particular, the extended portions 41b of the pad parts 41 are disposed near the elastic seal body 5. The extended portions 41b are respectively covered by the solder parts 26 for electric connection with the corresponding terminal parts 24. However, if a corrosive component (sulfur component such as sulfur gas) is present near the extended portions 41b, the pad parts 41 (extended portions 41b) containing copper are corroded by the corrosive component with time. The detection element assembly 2 is mounted in a range S surrounded by an alternate long and short dash line in FIG. 3.

### OTHER EMBODIMENTS

The present invention is not limited to the embodiments described above and illustrated by the drawings, and the following embodiments are also included in the technical scope of the present invention, for example.

(1) In Embodiment 1, on an outer side mounting surface 4a1 in a range outside the annular elastic seal body 5 in the mounting surface 4a of the circuit board 4, a coating layer (protective layer) is formed for protecting the outer side mounting surface 4a1 so as to prevent moisture, corrosive gases, or the like from contacting with the outer side mounting surface 4a1. As the coating layer, a known substance including polyolefin resin is used, for example. The coating layer is formed so as to cover the pattern-like wiring (containing copper) or the like formed on the circuit board 4. Since a corrosive component containing sulfur is not generated from the elastic seal body 5, the formation of the coating layer may be omitted on the outer side mounting surface 4a1 and the pattern-like wiring or the like may be exposed in another embodiment.

(2) The sulfur-free condensation silicone resin used for the elastic seal body is preferably one from which low molecular weight siloxanes are restrained from being generated. When a thermal conductivity gas sensor element is used as a gas sensor element, problems of deterioration due to low molecular weight siloxanes do not arise. Thus, a sulfur-free condensation silicone resin that generates low molecular weight siloxanes to some extent can be used.

### REFERENCE SIGNS LIST

1: gas sensor
2: detection element assembly
21: gas sensor package
22: gas sensor element
23: body part
24: terminal part
25: protective cap
26: solder part
3: casing
3A: first casing part
3B: second casing part
3B1: wall portion of second casing part
3B2: gas introduction port
4: circuit board
41: pad part
5: elastic seal body
7: measurement chamber

1. (canceled)

2. (canceled)

3. A gas sensor comprising:
a gas sensor element;
a box-like casing having a wall portion provided with a gas introduction port;
a circuit board having a mounting surface on which the gas sensor element is mounted, and disposed inside the casing such that the mounting surface is away from the wall portion and the gas sensor element overlaps with the gas introduction port; and
an annular elastic seal body formed on the mounting surface so as to surround the gas sensor element, and disposed between the mounting surface and the wall portion, wherein
the elastic seal body is made of sulfur-free condensation silicone resin and is adhered to the mounting surface and the wall portion.

4. The gas sensor according to claim 3, wherein the gas sensor element is composed of a thermal conductivity gas sensor element including a heating resistor having a resistance value that changes according to change in a temperature of the gas sensor element.
